(19) United States
(12) Patent Application Publication
(10) Pub. No.: US 2024/0024209 A1
(43) Pub. Date: Jan. 25, 2024

(54) HYDRATING COMPOSITION COMPRISING POLYMERS AND A COMPOUND CHOSEN FROM MENTHOL AND ITS DERIVATIVES

(71) Applicant: L'OREAL, Paris (FR)

(72) Inventors: Sabine ROUX, CHEVILLY LARUE (FR); Caroline SIRICHANDRA, CHEVILLY LARUE (FR); Maïssa-Hado FARROUX, CHEVILLY LARUE (FR)

(21) Appl. No.: 18/255,498

(22) PCT Filed: Dec. 10, 2021
(86) PCT No.: PCT/EP2021/085158

§ 371 Date: Jun. 1, 2023

(30) Foreign Application Priority Data
Oct. 12, 2020 (FR) 2012974

(51) Int. Cl. A61K 8/06; A61K 8/73; A61Q 19/00

(52) U.S. Cl. CPC A61K 8/062; A61K 8/732; A61Q 19/00; A61K 2800/54

(57) ABSTRACT

The invention relates to a composition in oil-in-water emulsion form comprising the following in a physiologically acceptable medium:
at least one polymer chosen from glyceryl polymethacrylates,
at least one superabsorbent polymer chosen from starches grafted by an acrylic polymer, and
at least one compound chosen from menthol and its derivatives.

The present invention relates to a composition, particularly cosmetic, in the form of an oil-in-water (O/W) emulsion that is easy to apply, non-tacky and non-greasy on application, with a fresh sensation and a long-term hydrating efficacy.

The skin is a tissue in which cells are contiguous and firmly attached to each other. Skin tissue forms an external coating comprising sebaceous or sudoriferous glands, and hair follicles. The skin, and particularly the scalp, are continuously renewed epithelia. Renewal, or desquamation, is a coordinated and finely regulated process leading to the elimination of surface cells, insensibly and invisibly.

The human skin is composed of two compartments, namely a surface compartment (the epidermis) and a deep compartment (the dermis).

The epidermis is conventionally divided into a base layer of keratinocytes forming a germinative layer of the epidermis, a layer called the spinous layer composed of several layers of polyhedric cells arranged on the germinative layers, one to three layers called the stratum granulosum composed of flattened cells containing distinct cytoplasmic inclusions, the keratohyalin grains and finally, a set of upper layers called corneal layers (or stratum corneum), composed of keratinocytes at the terminal stage of their differentiation called corneocytes.

Corneocytes are anucleate cells composed principally of a fibrous material containing cytokeratins, surrounded by a corneal envelope. There is permanent production of new keratinocytes to compensate for the continuous loss of epidermal cells in the stratum corneum according to a mechanism called desquamation.

However, an unbalance between the production of cells in the base layer and the desquamation rate can lead to the formation of scales on the skin surface. Similarly, for various reasons, a deficit of terminal differentiation of cells in the stratum corneum can lead to the formation of large, thick clumps of cells, visible to the naked eye and called “dander”, or in other situations, to thinning of the stratum corneum. This can cause fragility of the barrier properties of the epidermis, chronic dehydration of the stratum corneum, loss of mechanical elasticity, tightness, and make the skin lose its luster and transparency. Among examples of factors conducive to this alteration of the surface quality of the skin, mention may be made of stress, the winter period, excess sebum, a hydration disorder; this can also be the case for dry skin of elderly subjects.

Consequently, there is a need for compositions improving the hydration condition of the skin, avoiding tightness and feelings of discomfort during their application on the skin.

The inventors have now discovered that the association of a glyceryl poly(meth)acrylate, a starch grafted by an acrylic polymer and a compound derived from menthol, makes it possible to hydrate the skin in a satisfactory manner throughout the day and give it a fresh sensation, while being non-greasy and non-tacky on application.

Thus, the purpose of the present invention is a composition in oil-in-water emulsion form comprising the following in a physiologically acceptable medium:
At least one polymer chosen from glyceryl poly(meth)acrylates,
At least one superabsorbent polymer chosen from starches grafted by an acrylic polymer, and
At least one compound chosen from menthol and its derivatives.

The composition according to the invention is preferably cosmetic.

“Physiologically acceptable” means a medium compatible with keratin materials.

The present invention also relates to a cosmetic method for cleansing keratin materials, preferably the skin, comprising the application of a composition according to the invention on said keratin materials.

Another purpose of this invention is cosmetic use of a composition according to the invention to hydrate the skin.

Polymer Chosen from Glyceryl poly(meth)acrylates

The composition according to the present invention comprises at least one polymer chosen from glyceryl poly(meth)acrylates.

This polymer is water-soluble, has thickening and hydrating properties, and offers a light and non-tacky feel.

Preferably, it is chosen from glyceryl polyacrylates and glyceryl polymethacrylates. Advantageously, this polymer in 10% solution in water has a viscosity, as measured at 25° C. with a BROOKFIELD RTV viscometer, with a spindle 5 at 20 rpm, between 10000 and 20000 mPa·s, preferably between 12000 and 18000 mPa·s.

Preferably, glyceryl polymethacrylate is chosen. More particularly, glyceryl polymethacrylate is present in mixture form in a humectant, preferably glycerin.

According to the invention, the glyceryl polymethacrylate sold under the trade name “LUBRAJEL MS” by GUARDIAN INC or the glyceryl polyacrylate sold under the trade name “HISPAGEL 200 NS” by BASF can for example be used.

The polymer chosen from glyceryl poly(meth)acrylates is present preferably at an active substance content between 0.01% and 5% by weight of the composition, preferably between 0.05% and 3% by weight, preferably between 0.1% and 1% by weight.

Superabsorbent Polymer Chosen from Starches Grafted by an Acrylic Polymer

The composition according to the present invention comprises at least one superabsorbent polymer chosen from starches grafted by an acrylic polymer.

A “superabsorbent polymer” means a polymer that in its dry state, is capable of spontaneously absorbing at least 20 times its own weight of aqueous fluid, and particularly water and especially distilled water. Such superabsorbent polymers are described in the book “Absorbent polymer technology, Studies in polymer science 8” by L. BRAN NON-PAPPAS and R. HARLAND, published by Elsevier, 1990.

These polymers have high capacity for the absorption and retention of water and aqueous fluids. After absorption of the aqueous liquid, the polymer particles thus soaked in aqueous fluid remain insoluble in the aqueous fluid and thus retain their individualized particulate state.

The superabsorbent polymer can have a water absorption capacity varying from 20 to 2000 times its own weight (namely 20 g a 2000 g of water absorbed per gram of absorbent polymer), preferably from 30 to 1500 times, and more preferably from 50 to 1000 times. These water absorption characteristics are defined under normal temperature (25° C.) and pressure (760 mm Hg equal to 100000 Pa) conditions and for distilled water. The value of the water absorption capacity of a polymer can be determined by dispersing 0.5 g of polymer(s) in 150 g of a solution of water, waiting for 20 minutes, filtering the solution not absorbed on a 150 μm filter for 20 minutes and weighing the non-absorbed water.

The superabsorbent polymer used in the composition of the invention is in the form of particles. Preferably, the superabsorbent polymer, in the dry or non-hydrated state, has an average size less than or equal to 100 μm, preferably less than or equal to 50 μm, varying for example from 1 to 100 μm, preferably from 5 to 50 μm and more preferably from 7 to 30 μm.

The average size of particles corresponds to the average mass diameter (D50) measured by laser size grading or another equivalent method known to a person skilled in the art. These particles, once hydrated, swell by forming soft particles with an average size that can vary from 10 μm to 1000 μm.

Preferably, the superabsorbent polymers used in this invention are in the form of spherical particles.

The superabsorbent polymers according to the invention are starches grafted by an acrylic polymer. The acrylic polymer can be a homopolymer or a copolymer, particularly sodium polyacrylate. Such polymers have gelling properties and a feel similar to that of a silicone-containing compound (“silicone-like” feel).

Mention may particularly be made of starches grafted by an acrylic polymer and particularly by sodium polyacrylate, like those sold under the trade name Sanfresh ST-100MC by Sanyo Chemical Industries or Makimousse 25, Makimousse 12 or Makimousse 7 by Daito Kasei (INCI name Sodium polyacrylate Starch).

The superarbsorbent polymers used in this invention may or may not be cross-linked.

The superabsorbent polymers used in this invention are preferably starches grafted by an acrylic homo- or copolymer, particularly sodium polyacrylate, which are presented in particulate form.

Preferably, the superabsorbent polymer is chosen from starches grafted by an acrylic homopolymer, preferably in the form of particles with an average size (or average diameter) less than or equal to 100 microns, also preferably in the form of spherical particles.

The superabsorbent polymer chosen from the starches grafted by an acrylic polymer can be present in the composition according to the invention at an active material content ranging for example from 0.05% to 5% by weight, preferably from 0.1% to 3% by weight, preferably ranging from 0.2% to 2% by weight, preferably ranging from 0.3% to 1% by weight with respect to the total weight of the composition.

Compound Chosen from Menthol and its Derivatives

The composition according to the invention comprises at least one compound chosen from menthol and its derivatives.

This compound is preferably of formula (I):

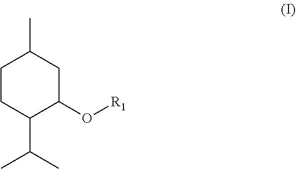

wherein, R1 denotes a hydrogen atom or a linear or branched, saturated or unsaturated, C1 to 12 alkyl radical, optionally substituted by one or more hydroxyl groups and one or more NRR′ groups where R and R′, identical or different, denote a hydrogen atom or a linear or branched, saturated or unsaturated, C1 to C4 alkyl radical.

If R1=H, then the compound of formula (I) is menthol.

Preferably, R1 denotes a C1 to C6, linear or branched, saturated or unsaturated alkyl radical, substituted by one or more hydroxyl groups. The C1 to C6, linear or branched, saturated or unsaturated alkyl radical, can be chosen from methyl, ethyl, n-propyl, i-propyl, n-butyl, i-butyl and n-hexyl radicals.

More preferably, R1 denotes a C1 to C6 linear and saturated alkyl radical, substituted by one or more hydroxyl groups, and preferably by one or two hydroxyl groups, preferably two hydroxyl groups. Preferably, R1 is the radical propane-1,2-diol.

Advantageously, the compound of formula (I) is menthoxypropanediol (also known as 3-L-menthoxy-1,2-propanediol).

This compound is particularly sold by Takasago under the name Coolact 10.

The compound chosen from menthol and its derivatives is present in the composition according to the invention in a quantity ranging from 0.01% to 5% by weight, more preferably from 0.05% to 1% by weight, and more preferably from 0.05% to 0.5% by weight, with respect to the total weight of the composition.

Aqueous Phase

The composition according to the invention comprises a physiologically acceptable aqueous phase. “Physiologically acceptable” means a medium compatible with keratin materials.

As the composition according to the invention is an O/W emulsion, this aqueous phase is the continuous phase.

The composition according to the invention preferably comprises an aqueous phase comprising water and optionally one or more organic solvent(s) soluble in water at 25° C.

These solvents can advantageously be chosen for example from linear or branched alkanols, in C2-C4, such as ethanol and isopropanol, propanol, butanol; polyols particularly with 3 to 20 carbon atoms, preferably 3 to 6 carbon atoms such as diglycerol, propyleneglycol, isoprene glycol, dipropyleneglycol, butylene glycol, hexylene glycol, 1,3-propanediol, pentylene glycol, polyethyleneglycols with 2 to 200 ethylene oxide units and mixtures thereof.

The composition generally comprises from 50% to 95% by weight of water with respect to the total weight of the composition, preferably from 60% to 90% by weight, preferably from 70% to 90% by weight.

The quantity of organic solvent(s) can range for example from 0.01% to 15% by weight, preferably from 0.5% to 13% by weight, more preferably from 1% to 10% by weight, more preferably from 3% to 10% by weight with respect to the total weight of the composition.

Preferably, the composition according to the invention is substantially free from alcohol (i.e. alkanol and/or polyol). “Substantially free” means that the composition comprises less than 0.5% by weight, preferably less than 0.3% by weight, preferably less than 0.1% by weight with respect to the total composition weight of alcohol. Preferably, the composition according to the invention is completely free from alcohol.

The quantity of aqueous phase can range from 50% to 99%, preferably from 60% to 95%, preferably from 65% to 90% by weight with respect to the total composition weight.

The composition generally comprises from 50% to 95% by weight of water with respect to the total weight of the composition, preferably from 60% to 90% by weight, preferably from 70% to 90% by weight.

Humectant

The composition according to the invention can comprise at least one humectant agent.

The humectant agent is preferably chosen from glycerol, betaine (trimethylglycine) and mixtures thereof.

The quantity of humectant(s) can range for example from 0.5% to 20% by weight, preferably from 1% to 15% by weight, more preferably from 2% to 12% by weight with respect to the total weight of the composition.

Surfactants

The composition according to the invention can also comprise at least one surfactant, preferably of HLB greater than or equal to 8.

Preferably, this surfactant is chosen from anionic surfactants, non-ionic surfactants and mixtures thereof.

Preferably, the surfactant is chosen from anionic surfactants. More preferably, the anionic surfactant is a citrate, such as glyceryl stearate citrate. Such a compound is sold under the name Axol C 62 Pellets by Degussa.

Preferably, the surfactant is a non-ionic surfactant. More preferably, the surfactant is a non-ionic surfactant chosen from the esters of fatty acid comprising 12 carbon atoms and of polyglycerol comprising 4 to 6 glycerol units.

The fatty acid and polyglycerol ester is formed from at least one acid comprising an alkyl or alkenyl chain containing 12 carbon atoms and 4 to 6 glycerol units.

According to one embodiment, the fatty acid and polyglycerol ester results from esterification of at least one saturated or unsaturated fatty acid, and a polyglycerol.

Preferably, the fatty acid and polyglycerol ester comprising 4 to 6 glycerol units is a mono- or diester, preferably a mono-ester.

The term “polyglycerol” denotes glyceryl polymers that are linear chains of glycerol units.

The esters that are more particularly considered according to the present invention are esters resulting from the esterification of polyglycerol and carboxylic acid(s) at C12, such as lauric acid.

The carboxylic acid may be linear or branched, saturated or unsaturated.

Preferably, it is a linear monocarboxylic acid.

In general, they are derived from the esterification of at least one hydroxyl function of a polyglycerol by a carboxylic acid at C12.

According to one particular embodiment, esters suitable for this invention can be derived from esterification of a polyglycerol by one or several identical or different carboxylic acids. It may be a hydroxylated mono-ester, a hydroxylated di-ester, a hydroxylated tri-ester, or a mixture thereof.

In a preferred embodiment of the invention, the ester is chosen from the esters of fatty acid comprising 12 carbon atoms and of polyglycerol comprising 5 glycerol units.

The esters of fatty acid comprising 12 carbon atoms and of polyglycerol comprising 5 glycerol units is formed (i) from at least one fatty acid comprising an alkyl or alkenyl chain containing 12 carbon atoms (also called C12 fatty acid), and (ii) from 5 glycerol units.

Preferably, the C12 fatty acid is saturated and contains a linear alkyl chain. Preferably, the C12 fatty acid is lauric acid.

In a preferred embodiment of the invention, the ester of fatty acid comprising 12 carbon atoms and of polyglycerol comprising 5 glycerol units is polyglyceryl monolaurate comprising 5 glycerol units, i.e. polyglyceryl-5 monolaurate.

A commercial product mostly based on polyglyceryl-5 monolaurate or PG-5 laurate is available under the trade name SUNSOFT A-121E-C® by TAIYO KAGAKU.

Especially, the inventors have discovered that polyglyceryl-5 monolaurate shows a very interesting effect on skin suppleness. Thus, said surfactant may be useful to increase skin suppleness and/or prevent or decrease skin tightness.

Thus preferably, the composition of the invention comprises at least polyglyceryl-5 monolaurate.

Preferably, the composition according to the invention comprises a mixture of at least one non-ionic surfactant as mentioned above, with at least one anionic surfactant, and preferably a citrate, such as glyceryl stearate citrate.

Preferably, the non-ionic surfactant is present in the composition according to the invention in a concentration ranging from 0.1% to 10% by weight, with respect to the total weight of the composition, preferably ranging from 0.25% to 5% by weight, and preferably ranging from 0.5% to 2% by weight.

Preferably, the anionic surfactant is present in the composition according to the invention in a concentration ranging from 0.01% to 5% by weight, with respect to the total weight of the composition, preferably ranging from 0.1% to 3% by weight, and preferably ranging from 0.3% to 2% by weight.

The total amount of surfactants can vary from 0.5% to 10% by weight, with respect to the total weight of the composition, preferably ranging from 0.7% to 5% by weight, and preferably ranging from 0.8% to 3% by weight.

The composition according to the invention comprises an oily phase dispersed in the aqueous phase: it is an O/W emulsion.

Oily Phase

The composition according to the invention also comprises at least one oil, particularly a cosmetic oil, forming an oily phase. It can further contain other fats.

By “oil”, it should be understood a non-aqueous compound, liquid at 25° C. and at atmospheric pressure (1.013×10⁵ Pa), not water-miscible.

By “not miscible”, it should be understood that the mixture of the same quantity of water and oil, after stirring, does not lead to a stable solution that comprises only one single phase, in the aforementioned conditions of temperature and pressure. The observation is made with the naked eye or using a phase contrast microscope if necessary, on 100 g of mixture obtained after Rayneri stirring sufficient to cause a vortex to appear within the mixture (for the purposes of information 200 to 1000 rpm); the resulting mixture being left to sit, in a closed bottle, for 24 hours at ambient temperature before observation.

By way of oils suitable for use in the composition according to the invention, mention may be made for example of:
hydrocarbon oils of plant origin, such as liquid fatty acid triglycerides having from 4 to 10 carbon atoms such as heptanoic or octanoic acid triglycerides or, for example, sunflower, corn, soybean, pumpkin, grape seed, sesame, hazelnut, apricot, macadamia, arara, sunflower, castor, avocado oils, caprylic/capric acid triglycerides such as those sold by Stearineries Dubois or those sold under the trade names Miglyol 810, 812 and 818 by Dynamit Nobel, jojoba oil, shea butter oil;
esters and synthetic esters, in particular fatty acids, such as oils having formulas R1COOR2 and R1OR2 wherein R1 is the residue of a fatty acid having 8 to 29 carbon atoms, and R2 is a hydrocarbon chain, optionally branched, containing 3 to 30 carbon atoms, such as for example Purcellin oil, isononyl isononanoate, cetearyl isononanoate (which is a mixture of C16 and C18 alkyl esters with isononanoic acid), isopropyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl malate, triisocetyl citrate; heptanoates, octanoates, decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate or dipentaerythrityl pentaisononanoate;
linear or branched hydrocarbons, whether mineral or synthetic, such as paraffin oils, whether volatile or not, and their derivatives, branched-chain hydrocarbon oils comprising 10 to 30 carbon atoms, such as isohexadecane, isododecane, isoparaffins and their mixtures, vaseline, polydecenes, polyisobutenes, hydrogenated polyisobutenes such as Parleam® sold by NIPPON OIL FATS, PANALANE H-300 E sold by AMOCO, VISEAL 20000 sold by SYNTEAL, REWOPAL PIB 1000 sold by WITCO, or PARLEAM LITE sold by NOF Corporation. Among the preferred linear or branched hydrocarbons of synthetic origin, mention can be made of squalane, which is an oil obtained from hydrogenating squalene;
partially hydrocarbon and/or silicone fluorinated oils such as those described in the document JP-A-2-295912;
silicone oils such as polymethylsiloxanes (PDMS), optionally volatile with a linear or cyclic silicone chain, liquid or pasty at ambient temperature, particularly cyclopolydimethylsiloxanes (cyclomethicones) such as cyclohexasiloxane; polydimethylsiloxanes comprising alkyl, alkoxy or phenyl pendant or silicon chain-end groups, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes; or
mixtures thereof.

Preferably, the composition according to the invention is substantially free from silicone oil. “Substantially free” means that the composition comprises less than 0.5% by weight, preferably less than 0.3% by weight, preferably less than 0.1% by weight with respect to the total composition weight of silicone oil. Preferably, the composition according to the invention is completely free from silicone oil.

Preferably, the composition according to the invention is substantially free from mineral oil. “Substantially free” means that the composition comprises less than 0.5% by weight, preferably less than 0.3% by weight, preferably less than 0.1% by weight with respect to the total composition weight of mineral oil. Preferably, the composition according to the invention is completely free from mineral oil.

Preferably, the composition according to the invention comprises at least one oil chosen from the oils of formulas R1COOR2 and R1OR2 wherein R1 represents the residue of a fatty acid including 8 to 29 carbon atoms, and R2 represents an optionally branched hydrocarbon chain, containing 3 to 30 carbon atoms, linear or branched hydrocarbons of synthetic origin and mixtures thereof.

As indicated hereinabove, the composition according to the invention may comprise other fatty substances in the oily phase. Preferably, this fatty substance is chosen from pasty fatty substances.

For the purposes of the invention, the term “pasty fatty substance” refers to a lipophilic fatty compound having a reversible solid/liquid change of state, having an anisotropic crystalline organization in the solid state, and comprising a liquid fraction and a solid fraction at a temperature of 23° C. In other words, the initial melting point of the pasty compound may be less than 23° C. The liquid fraction of the pasty compound measured at 23° C. may represent 9 to 97% by weight of the compound. This liquid fraction at 23° C. preferably represents between 15 and 85%, more preferably between 40 and 85% by weight.

The melting point of a solid fatty substance can be measured using a differential scanning calorimeter (DSC), for example the calorimeter sold under the trade name “DSC Q100” by TA Instruments with the “TA Universal Analysis” software, according to the protocol defined hereinabove. The liquid fraction by weight of the pasty compound at 23° C. is more particularly equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is said to be in the solid state when the entire mass thereof is in solid crystalline form. The pasty compound is said to be in the liquid state when the entire mass thereof is in liquid form. The enthalpy of fusion of the pasty compound is in particular equal to the area under the curve of the thermogram obtained using a differential scanning calorimeter. The enthalpy of fusion of the pasty compound is the quantity of energy required to change the compound from the solid state to the liquid state. It is expressed in J/g. The enthalpy of fusion consumed at 23° C. is the quantity of energy required by the sample to change from the solid state to the state presented at 23° C. consisting of a liquid fraction and a solid fraction.

The pasty compound(s) can be in particular chosen from synthetic pasty compounds and fatty substances of plant origin. The pasty compound(s) can be in particular chosen from:
lanolin and its derivatives, such as lanolin alcohol, oxyethylenated lanolins, acetylated lanolin, lanolin esters such as isopropyl lanolate, oxypropylenated lanolins;
polyol ethers chosen from C2-C4 pentaerythritol and polyalkylene glycol ethers, fatty alcohol and sugar ethers, and mixtures thereof. For example, mention can be made of pentaerythritol and polyethylene glycol ether comprising 5 oxyethylene patterns (5 OE) (CTFA name: PEG-5 Pentaerythrityl Ether), pentaerythritol and polypropylene glycol ether comprising 5 oxypropylene units (5 OP) (CTFA name: PPG-5 Pentaerythrityl Ether), and the mixtures thereof and more specifically the mixture of PEG-5 Pentaerythrityl Ether, PPG-5 Pentaerythrityl Ether and soybean oil, sold under the name “Lanolide” by VEVY, wherein the ratio of the constituents by weight is 46:46:8:46% PEG-5 Pentaerythrityl Ether, 46% PPG-5 Pentaerythrityl Ether and 8% soybean oil,
polyethers derived from polyetherification between one or a plurality of C2-C100, preferably C2-050, diols. Of the liposoluble polyethers, ethylene-oxide and/or propylene-oxide copolymers with C6-C30 long-chain alkylene-oxides are particularly considered, more preferably such that the weight ratio of ethylene-oxide and/or propylene-oxide with alkylene-oxides in the copolymer is 5:95 to 70:30. In this family, particular mention may be made of copolymers such as long-chain alkylene-oxides arranged in blocks having a mean molecular weight of 1000 to 10000, for example a polyoxyethylene/polydodecyl glycol block copolymer such as the dodecanediol (22 mol) and polyethylene glycol (45 EO) ethers marketed under the brand ELFACOS ST9 by AKZO NOBEL.
oligomer glycerol esters, especially the esters of diglycerol, with monocarboxylic acids, possibly hydroxylated, linear or branched, saturated or not, preferably saturated, C6-C20, and/or dicarboxylic acids, linear or branched, saturated or not, preferably saturated, C6-C10, in particular condensates of adipic acid and diglycerol, for which a portion of the hydroxyl groups of the glycerols have reacted with a mixture of fatty acids, such as stearic acid, capric acid, stearic acid, isostearic acid and 12-hydroxystearic acid, such as for example bis-diglyceryl polyacyladipate-2 sold under the reference SOFTISAN® 649 by Sasol,
butters of plant origin, such as mango butter, such as the one sold under the reference Lipex 203 by AARHUSKARLSHAMN, shea butter, in particular the one of which the INCI name is Butyrospermum Parkii Butter, such as the one sold under the reference Sheasoft® by AARHUSKARLSHAMN, cupuacu butter (Rain Forest RF3410 from Beraca Sabara), murumuru butter (RAIN FOREST RF3710 from Beraca Sabara), cocoa butter, babassu butter such as the one sold under the name Cropure Babassu SS-(LK) by Croda, as well as orange wax such as for example the one sold under the reference Orange Peel Wax by Koster Keunen,
totally or partially hydrogenated plant oils, such as for example hydrogenated soybean oil, hydrogenated coconut oil, hydrogenated rapeseed oil, mixtures of hydrogenated plant oils such as the mixture of hydrogenated plant oils of soybean, coconut, palm and rapeseed, for example the mixture sold under the reference Akogel® by AARHUSKARLSHAMN (INCI name Hydrogenated Vegetable Oil), trans isomerized partially hydrogenated jojoba oil manufactured or sold by Desert Whale under the commercial reference Iso-Jojoba-50®, partially hydrogenated olive oil such as, for example, the compound sold under the reference Beurrolive by Soliance,
hydrogenated castor oil esters, such as dimer dilinoleate hydrogenated castor oil for example RISOCAST-DA-L sold by KOKYU ALCOHOL KOGYO, hydrogenated castor oil isostearate for example SALACOS HCIS (V-L) sold by NISSHIN OIL,
and mixtures thereof.

Preferably, the composition according to the invention comprises, in the oily phase, at least one pasty fatty substance chosen from butters of plant origin.

The quantity of oily phase can range from 1% to 20%, preferably from 2% to 15%, preferably from 3% to 10% by weight with respect to the total composition weight.

The composition according to the invention may also comprise any additive usually used in cosmetics, such as in particular fillers, dyes, UV filters, preservatives, and/or active ingredients.

As a UV filter, mention may be made of organic or mineral UV filters, well known to those skilled in the art.

As examples of dyes, mention may be made of the dyes chosen from water-soluble colorants, powdery dyes such as pigments, nacres and glitters well known to those skilled in the art.

Preferably, the composition according to the invention is substantially free from fillers. “Substantially free” means that the composition comprises less than 0.5% by weight, preferably less than 0.3% by weight, preferably less than 0.1% by weight with respect to the total composition weight of filler. Preferably, the composition according to the invention is completely free from filler.

Finally, the invention also relates to a cosmetic method for cleansing keratin materials, preferably the skin, comprising the application of a composition according to the invention.

It also relates to the cosmetic use of a composition according to the invention to hydrate the skin.

Concrete, yet non-limiting, examples, illustrating the invention, will now be provided.

In the examples, the temperature is ambient temperature (20° C.) expressed in degrees Celsius unless mentioned otherwise, and the pressure is atmospheric pressure, unless mentioned otherwise.

In the examples, quantities of the ingredients of the compositions are given as a by weight relative to the total weight of the composition (% w/w).

### EXAMPLE 1: PREPARATION OF A COMPOSITION ACCORDING TO THE INVENTION AND EVALUATION OF THIS COMPOSITION COMPARED TO COMPARATIVE COMPOSITIONS

The composition F1 according to the invention of Table 1 below is prepared with the following protocol:

The ingredients of phase A1 are mixed together, and heated to 65-70° C. to solubilize the hydroxyacetophenone.

The gelling agent of phase A2 is added, to obtain phase A.

The ingredients of phase B are mixed by heating to 65° C.

Phase B is emulsified in phase A, to obtain a white emulsified gel. Finally, phase C is added.

Comparative compositions F2 to F4 (marked with an asterisk) are prepared according to the same protocol.

Each composition is then evaluated for sensory properties by a panel of 3 people analyzing the application of each composition on the skin as well as the finish on the skin.

The acceptability of the compositions is defined by the hydration, finish on the skin, and the fresh sensation.

TABLE 1

| | | F1 | F2* | F3* | F4* |
|---|---|---|---|---|---|
| Phase | Ingredient | (% w/w) | (% w/w) | (% w/w) | (% w/w) |
| A2 | GLYCERYL POLYACRYLATE | 10 | 10 | 10 | — |
| | (Hispagel 200 NS from BASF) | (*0.4% a.s.) | (*0.4% a.s.) | (*0.4% a.s.) | |
| B | SODIUM POLYACRYLATE | 0.75 | — | 0.75 | 0.75 |
| | STARCH (Makimousse 12 from | | | | |
| | Daito Kasei) | | | | |
| B | MENTHOXYPROPANEDIOL | 0.1 | 0.1 | — | 0.1 |
| | (Coolact 10 from Takasago) | | | | |
| A1 | BETAINE | 2 | 2 | 2 | 2 |
| B | POLYGLYCERYL-5 LAURATE | 1 | 1 | 1 | 1 |
| B | GLYCERYL STEARATE CITRATE | 0.5 | 0.5 | 0.5 | 0.5 |
| B | PRESERVATIVE | Qs | Qs | Qs | Qs |
| A1 and C | Water | QSP 100 | QSP 100 | QSP 100 | QSP 100 |
| B | CETEARYL ISONONANOATE | 1.5 | 1.5 | 1.5 | 1.5 |
| B | SQUALANE | 1.5 | 1.5 | 1.5 | 1.5 |
| B | Shea butter | 2 | 2 | 2 | 2 |
| | Evaluation of the sensory | Soft, fresh, | Lack of | Lack of | Lack of |
| | properties of the formulas | emollient, non- | softness on | freshness on | emollience |
| | | greasy and | application. | application and | and |
| | | non-tacky | Insufficiently | throughout the | hydration |
| | | application. | soft, slightly | day. | on |
| | | Feeling of | greasy and | | application |
| | | hydration and | tacky skin | | and |
| | | freshness | finish. | | throughout |
| | | throughout the | More flexible | | the day. |
| | | day | texture. | | |
| | Formula acceptability | ACCEPTABLE | NOT | NOT | NOT |
| | | | ACCEPTABLE | ACCEPTABLE | ACCEPTABLE |

*a.s. = active substance

Conclusion: only formula F1 according to the invention comprising glyceryl polyacrylate, sodium polyacrylate starch and menthoxypropanediol provides hydration and freshness throughout the day, while being non-greasy and non-tacky on application.

Thus, composition F1 according to the invention makes it possible to hydrate the skin in a satisfactory manner throughout the day and give it a fresh sensation, while being non-greasy and non-tacky on application.

1. A composition in the form of an oil-in-water emulsion comprising in a physiologically acceptable medium:
At least one polymer chosen from glyceryl poly(meth)acrylates,
At least one superabsorbent polymer chosen from starches grafted by an acrylic polymer, and
At least one compound chosen from menthol and its derivatives.

2. The composition according to claim 1, characterized in that glyceryl poly(meth)acrylate is present at an active substance content between 0.1% and 5% by weight of the composition.

3. The composition according to claim 1, characterized in that the superabsorbent polymer is a starch grafted by an acrylic homo- or copolymer which is presented in particulate form.

4. The composition according to claim 1, characterized in that the superabsorbent polymer is present at an active substance content ranging from 0.05% to 5% by weight with respect to the total weight of the composition.

5. The composition according to claim 1, characterized in that the compound chosen from menthol and its derivatives is of formula (I): 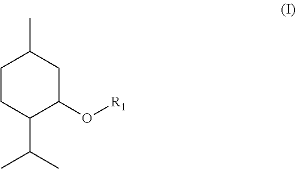
wherein, R1 denotes a hydrogen atom or a linear or branched, saturated or unsaturated, C1 to 12 alkyl radical, optionally substituted by one or more hydroxyl groups and one or more NRR′ groups where R et R′, identical or different, denote a hydrogen atom or a linear or branched, saturated or unsaturated, C1 to C4 alkyl radical.

6. The composition according to claim 1, characterized in that the compound chosen from menthol and its derivatives is menthoxypropanediol.

7. The composition according to claim 1, characterized in that the compound chosen from menthol and its derivatives is present in an amount ranging from 0.01% to 5% by weight with respect to the total weight of the composition.

8. The composition according to claim 1, characterized in that it comprises at least one humectant agent.

9. The composition according to claim 1, characterized in that it comprises at least one surfactant.

10. The composition according to claim 1, characterized in that it comprises at least polyglyceryl-5 monolaurate.

11. The composition according to one of the claim 1, characterized in that it comprises at least one oil chosen from the oils of formulas R1COOR2 and R1OR2 wherein R1 represents the residue of a fatty acid including 8 to 29 carbon atoms, and R2 represents an optionally branched hydrocarbon chain, containing 3 to 30 carbon atoms, linear or branched hydrocarbons of synthetic origin and mixtures thereof, and optionally further at least one pasty fatty substance.

12. The composition according to claim 1, characterized in that it is substantially free from silicone oil, and/or substantially free from mineral oil, and/or substantially free from filler.

13. A method for cosmetic care of keratin materials comprising application of a composition according to claim 1 on said keratin materials.

14. A method for hydrating skin, comprising application of a composition according to claim 1 on said skin.

15. The composition according to claim 2, characterized in that the superabsorbent polymer is a starch grafted by an acrylic homo- or copolymer which is presented in particulate form.

16. The composition according to claim 2, characterized in that the superabsorbent polymer is present at an active substance content ranging from 0.05% to 5% by weight with respect to the total weight of the composition.

17. The composition according to claim 3, characterized in that the superabsorbent polymer is present at an active substance content ranging from 0.05% to 5% by weight with respect to the total weight of the composition.

18. The composition according to claim 2, characterized in that the compound chosen from menthol and its derivatives is of formula (I): 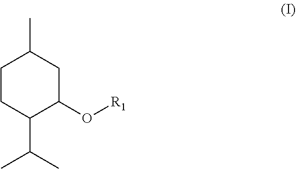
wherein, R1 denotes a hydrogen atom or a linear or branched, saturated or unsaturated, C1 to 12 alkyl radical, optionally substituted by one or more hydroxyl groups and one or more NRR′ groups where R et R′, identical or different, denote a hydrogen atom or a linear or branched, saturated or unsaturated, C1 to C4 alkyl radical.

19. The composition according to claim 3, characterized in that the compound chosen from menthol and its derivatives is of formula (I): 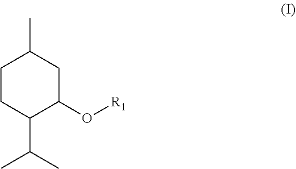
wherein, R1 denotes a hydrogen atom or a linear or branched, saturated or unsaturated, C1 to 12 alkyl radical, optionally substituted by one or more hydroxyl groups and one or more NRR′ groups where R et R′, identical or different, denote a hydrogen atom or a linear or branched, saturated or unsaturated, C1 to C4 alkyl radical.

20. The composition according to claim 4, characterized in that the compound chosen from menthol and its derivatives is of formula (I): 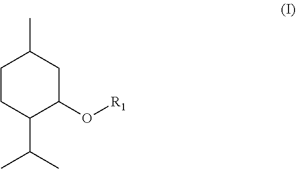
wherein, R1 denotes a hydrogen atom or a linear or branched, saturated or unsaturated, C1 to 12 alkyl radical, optionally substituted by one or more hydroxyl groups and one or more NRR′ groups where R et R′, identical or different, denote a hydrogen atom or a linear or branched, saturated or unsaturated, C1 to C4 alkyl radical.
